(19) United States
(12) Patent Application Publication
(10) Pub. No.: US 2016/0138090 A1
(43) Pub. Date: May 19, 2016

(54) DETERMINATION OF INTRACELLULAR BACTERIA

(71) Applicant: STENDER DIAGNOSTICS, Gentofte (DK)

(72) Inventor: Henrik Stender, Gentofte (DK)

(21) Appl. No.: 14/898,860

(22) PCT Filed: Jun. 12, 2014
(86) PCT No.: PCT/DK2014/000032

§ 371 Date: Dec. 16, 2015

(30) Foreign Application Priority Data
Jun. 16, 2013 (DK) PA 2013 00361

(51) Int. Cl. C12Q 1/68

(52) U.S. Cl. CPC C12Q 1/689; C12Q 1/6841

(57) ABSTRACT

This application pertains to methods for the whole-cell analysis of intracellular bacteria. The methods are capable of making a determination of whether or not a sample (e.g. a clinical sample) comprises one or more select bacteria within host cells, for example, predatory host cells such as phagocytic cells. The method is performed on substantial intact bacteria and may be performed without the use of permeabilising or lysis reagents and using PNA probes. Furthermore, the application pertains to in situ hybridization methods for Gram-positive bacteria performed using buffered saline for hybridization.

### RELATED APPLICATIONS

This application claims the benefit of Provisional Application PA 2013 00361 filed Jun. 16, 2013. The entire content of this application is incorporated herein by this reference in its entirety.

### FIELD

This application relates to the field of determination of intracellular bacteria and, in some embodiments, pertains to the analysis of bacteria within phagocytic cells.

### BACKGROUND

Traditional methods for analysis of bacteria rely on extracellular analysis where intracellular bacteria are either released into the extracellular environment or where intracellular bacteria are separated or excluded. Furthermore, traditional methods typically require culturing of the bacteria, such that results are not available the same day. The analysis is further complicated when the host cells are predatory cells, such as phagocytic cells, degrading the intracellular bacteria. Conditions where bacteria are predominately found intracellular are thus inherently difficult to diagnose by traditional methods. Examples include diagnosis of sepsis or bloodstream infections where extracellular bacteria are cleared by either phagocytosis or antibiotic therapy potentially resulting in false negative results by traditional methods.

Examples of methods for determination of intracellular bacteria comprises conventional staining methods, such as acridine orange, which can detect bacteria, but only provides presumptive information, such as morphology, about the bacteria and thus of limited clinical value (Int J Biol Med Res. 2: 360-368; Pejouhesh 31:155-158). Methods providing identification of intracellular bacteria have also been described, such as determination of intracellular bacteria in polymorphonuclear leukocytes (PMN) by in situ hybridization, which have been described by Matsuhisa et al., (Biotech Histochem. 69:31-7; Microbiol. Immunol. 38:511-517; U.S. Pat. No. 5,358,846; U.S. Pat. No. 7,651,837). This method uses enzymes for permeabilization of host cells and lysis of bacteria, respectively, to make the bare bacterial target molecules assessable, and biotinylated probes in hybridization buffer containing formamide (toxic) and required overnight incubation, and signal amplification using enzyme-labeled streptavidin.

While the literature does contain various reports related to determining intracellular bacteria, these assays are hampered by the use of complex and time-consuming permeabilization and/or lysis steps, signal amplification steps and prolonged hybridization steps using toxic chemicals and/or are not able to further characterize the bacteria beyond morphology. Simple in situ hybridization methods have been applied for analysis of bacteria from blood cultures (J Clin Microbiol 40:247-251), however, the methods are performed on cultured bacteria and thus—like traditional method described above—require time-consuming culturing step and exclude non-viable bacteria within phagocytic cells (Clin Perinatol 37:411-419). There is therefore a need for faster, simpler and less toxic methods for determination of intracellular bacteria within the host cells.

### SUMMARY OF THE INVENTION

Is has surprisingly been discovered that intracellular bacteria can be determined while keeping both the host cell and the intracellular bacteria substantially intact and that the various methods disclosed herein may thus be performed without performing cell permeabilization or lysis step(s). In particularly, it has been discovered that the whole cells analysis using bacteria-directed probes can be performed where the bacteria-directed probes are penetrating first the host cell containing intracellular bacteria and then the bacteria without the use of permeabilization or lysis reagents. Keeping the bacteria substantially intact for whole cells analysis has the advantage of the target molecules being concentrated within the bacteria and thus allow for analysis without the use of signal or target amplification techniques.

In addition, it has been shown that methods disclosed herein can be applied for determination of intracellular bacteria in phagocytoic host cells.

It has also been discovered that whole cell analysis methods for bacteria, including Gram-positive bacteria, such as S. aureus, may be performed without the use of toxic denaturing chemicals, such as formamide, commonly used for hybridization buffer and in fact may be performed using saline buffers.

By combining whole cell analysis of bacteria with intracellular analysis the methods disclosed are using the bacteria to retain the target molecules and the host cells containing the bacteria to retain the bacteria hereby providing several advantages over current methods. For example, this concept offers orders of magnitudes higher target concentration compared to the target concentration as if the host cells and/or bacteria were lysed and/or target released into the whole sample volume hereby providing basis for higher sensitivity. As another example, the analysis of substantially intact bacteria reduces the risk of false-positive results by not detecting bare target molecule and by not detecting degraded bacteria potentially reflecting past infection or contamination hereby providing basis for higher sensitivity.

In summary, the invention offers significant improvements over prior art in the form of being faster, simpler, safer, more sensitive and/or more specific and may thus find great utility as a clinical assay in hospitals or other applications where intracellular bacteria are found. For example, the present invention enable analysis of bacteria within the phagocytic cells in blood without diluting with blood culture medium, i.e. the intracellular bacteria are kept within the phagocytic cells and the bacteria are not multiplied. Optionally, the phagocytic cells are isolated, for example by centrifugation, hereby concentrating the bacteria. This way identification of bacteria in blood can be performed faster as there is no need for multiplication by over-night incubation. Furthermore, non-viable bacteria, for example bacteria from patients being treated with antibiotics preventing bacteria from multiplying in blood culture media, may be identified.

### BRIEF DESCRIPTION OF THE FIGURES

FIG. 1. Left fluorescence microscope image (A) shows blue cocci within blue PMN. Right fluorescence microscope image (B) shows green cocci identified by PNA probes whereas PMN is non-fluorescent/weak reddish background. The blue cocci observed within the PMN have same morphology and position as the green cocci.

FIG. 2. Left fluorescence microscope image (A) shows S. aureus as bright green cocci. Right fluorescence microscope image (B) shows S. epidermidis as none/weak-fluorescent cocci.

### DETAILED DESCRIPTION OF THE INVENTION

### General

It is to be understood that the discussion set forth below in this “General” section can pertain to some, or to all, of the various embodiments of the invention described herein.

Labels:

Non-limiting examples of labels (i.e. detectable moieties or markers) suitable for labeling probes used in the practice of this invention include a bead, chromophore, a fluorophore, a spin label, a radioisotope, an enzyme, a hapten, a chemiluminescent compound, a quantum dot or combinations of two or more of the foregoing. Some examples of haptens include 5(6)-carboxyfluorescein, 2,4-dinitrophenyl, digoxigenin, and biotin. Some examples of fluorochromes (fluorophores) include 5(6)-carboxyfluorescein (Flu), 6-((7-amino-4-methylcoumarin-3-acetyl)amino)hexanoic acid (Cou), 5(and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye.

Independently Detectable Labels/Multiplex Analysis:

In some embodiments, a multiplex method (assay) is performed. In a multiplex assay, numerous conditions of interest are simultaneously or sequentially examined. Multiplex analysis relies on the ability to sort sample components or the data associated therewith, during or after the assay is completed. A multiplex assay (as used herein) commonly relies on use of two or more uniquely identifiable probes.

In a multiplex assay, one or more distinct independently detectable labels (typically each distinct label (or a distinct combination of labels) is linked to a different probe) are used to uniquely mark (i.e. stain) two or more different bacteria of interest. In some cases, two (or more) unique labels may be directed to the same bacteria thereby generating a unique stain that results from the presence of the two (or more) unique labels in the bacteria. The ability to differentiate between and/or quantify each of the uniquely stained bacteria provides the means to multiplex the assay because the data that correlates with each uniquely marked (i.e. stained) bacteria can be correlated with a condition or conditions sought to be determined (e.g. select bacteria).

Methods can be multiplexed in many ways and multiplexing is limited only by the number of independently detectable labels (or independently detectable probes) that can be used or detected in an assay. For example, some assays may be designed to detect and identify the presence of several (e.g. two, three, four, five, six or more) different bacteria in a sample.

Whole-Cell Assays:

Methods disclosed herein involve whole-cell assays. Whole-cell assays are performed on intact or substantially intact cells. Some examples of whole-cell assays are in-situ hybridization (ISH), fluorescence in-situ hybridization (FISH) and immunocytochemistry (ICC) assays. In some embodiments, a whole-cell assay is not strictly an ISH, FISH or ICC assay. For example, whole-cell assays may involve a combination of two or more of these different assay. More specifically, some embodiments of this invention contemplate use of oligomer (hybridization) probes used in combination with, for example, antibody probes.

ISH:

As used herein “in situ hybridization (ISH)” refers to methods practiced using a hybridization probe directed to a nucleic acid target. ISH may be carried out using a variety of detectable or detectably labeled probes capable of hybridizing to a cellular nucleic acid sequence. When fluorescently labeled probes are used, the technique is called FISH. The ISH probes may be labeled directly (e.g., by use of a covalently linked fluorescent-label) or indirectly (e.g., through a ligand-labeled antiligand system).

Immunocytochemistry (ICC):

As used herein, immunocytochemistry refers to the use of antibody or antibody fragments to stain bacteria of a sample through the interaction of an antibody probe (or antibody fragment probe) and an antigen within bacteria. The staining may occur by use of only primary antibodies or it may involve the use of (labeled) secondary antibodies. Hence, the antibody (or antibody fragment) probe can be directed to an antigen target that is specific for the select bacteria. The antibody probe can be labeled (i.e. direct detection) or the antibody probe/antigen target complex formed by the binding of the antibody probe to its respective antigen target within the bacteria can be determined by use of labeled secondary antibody that binds to said antibody probe/antigen target complex (i.e. indirect detection). No matter what is being targeted, at least one antibody is labeled with at least one detectable moiety such that when said labeled antibody binds, the bacteria is stained. Moreover, ICC can be combined with ISH or FISH procedures to thereby determine select bacteria according to the methods disclosed herein.

Samples:

Bacteria are everywhere. A sample comprising intracellular bacteria can come from any source. The source of a sample is not intended to be a limitation associated with the practice of any method disclosed herein. For example, samples can be environmental samples such as samples from soil or water. Samples can come from consumer staples such as food, beverages or cosmetics. Samples can come from crime scenes (e.g. for forensic analysis). Samples can come from war zones or from sites of a suspected terrorist attack. Samples can come from clinical sources. Samples from clinical sources can come from any source such as a human, a plant, a fish or an animal. Some non-limiting examples of clinical samples (from clinical sources) include blood, pus, sputum, spinal fluid, amniotic fluid, stool, urine, nasal swabs, throat swabs and the like. Samples can include samples prepared, or partially prepared, and/or fractionated for a particular analysis. For example, the sample may be a specimen that has been fixed and/or stored for a period of time.

Sample Fractionation:

Samples may be fractionated to separate or concentrate the cells optionally containing intracellular bacteria. For example, white blood cells may be separated from the blood for example by collecting the ‘buffy coat’ or other methods to purify, separate or concentrate the host cells. Furthermore, host cells may be further purified, separated or concentrated. For example, CD64 positive neutrophils may be purified, separated or concentrated to further enrich for host cells optionally containing intracellular bacteria.

Sample fractionation may also be virtual or visual using for example antibodies to selective stain host cells optionally containing intracellular cells, such that subsequent determination is performed on selectively stained host cells.

Intracellular Bacteria:

Bacteria may be found intracellular as part of their normal life cycle and/or bacteria may be found intracellular due to for example host deference mechanisms, i.e. by phagocytosis. Bacteria may also be other cellular microorganisms, such as spores or elementary bodies as longs they are cells inside a host cells, as well as mycete, protozoon, parasite or the like. Bacteria may include, for example, Staphylococcus, Pseudomonas, Enterococcus, Colibacillus, Streptococcus, Pneumococcus, Tubercle bacillus, Helicobacter pylori, Listeria, Yersinia, Brucellar or the like. Mycete may include, for example, Candida, Aspergillus, Actinomyces, Coccidioides, Blastomyces or the like. Protozoon may include, for example, Karyamoebina falcata, Trichomonas vaginalis, Malaria, Toxoplasma or the like. Parasite may include, for example, Trypanosoma or the like. In particular, the causative microorganisms of sepsis or bacteriemia may include, for example, Gram-positive bacteria of Staphylococcus genus (Staphylococcus aureus, Staphylococcus epidermidis) and Enterococcus genus (Enterococcus faecalis, Enterococcus faecium, Streptococcus pneumoniae, Streptococcus pyogenes, Streptococcus agalactiae), Gram-negative bacteria like enterobacteriaceae (Escherichia coli, Enterobacter cloacae, Klebsiella pneumonia), aerophilic rod of Pseudomonas genus (Pseudomonas aeruginosa).

Host Cell:

Host cells may be both cells containing bacteria as part of their normal life cycle, i.e. symbiosis and/or predatory host cells digesting or degrading bacteria due to for example host defense mechanisms, i.e. phagocytic cells, such as white blood cells, PMN or macrophages. The host cells are typically mammalian, human, or eukaryotic cells, but any cells containing another cell for any reason is a host cell.

Probes:

Unless expressly limited by specific language or discussion herein, any probe that can be used to determine a select bacteria based on selective binding of said probe to its respective target can be used in the practice of embodiments of this invention. In some embodiments, a probe can be an antibody or antibody fragment. In some embodiments, a probe can be a peptide or protein. A probe used in the practice of embodiments of this invention can be a nucleic acid (e.g. DNA or RNA), a nucleic acid analog (e.g. LNA), a nucleic acid mimic (e.g. PNA, PP or morpholino) or a chimera. In some embodiments, the probe or probes is/are 10 to 20 nucleobase subunits in length. Probes are described herein in terms of “nucleobase subunits in length” since only nucleic acids comprise nucleotides whereas all of these different oligomer types comprise one nucleobase per subunit. Probes used in embodiments of this invention can be prepared by denovo synthesis or by other methods.

It is to be understood that numerous probes exist in the biological arts for detecting specific bacteria. Consequently, the nature of the probe (for purposes of this invention) is not intended to be limited except as expressly disclosed herein.

In some embodiments, probes used in the practice of this invention can be unlabeled provided that there is an available mechanism for determining the probe/target complex formed by binding of the probe to its respective target. For example, an unlabeled (primary) antibody-based probe can be determined by use of a secondary detectably labeled antibody that binds to said unlabeled (primary) antibody-based probe (See for example: U.S. Pat. No. 6,524,798 at col. 3, lines 28-40 and U.S. Pat. No. 7,455,985 at col. 12, lines 12-63). For example, said unlabeled (primary) antibody-based probe may be used to determine the select bacteria. Thus, the complex (i.e. labeled secondary antibody/primary antibody/target complex) formed upon binding of all molecules can be determined (and hence the select bacteria) by determining said label of said secondary antibody. Other types of unlabeled probes can similarly be determined by use of a labeled molecule that selectively binds to said unlabeled probe or the complex formed by binding of said unlabeled probe to its respective target (See for example: U.S. Pat. No. 5,612,458).

In some embodiments, probes can be labeled with at least one detectable moiety (i.e. at least one label). In some embodiments, each probe will comprise only one label. In some embodiments, the probe or probes used to determine the select trait (e.g. methicillin-resistance) will comprise only one label. In some embodiments, mixtures of probes (e.g. mixtures of mRNA-directed probes) are used wherein each probe comprises one label or two labels (i.e. a mixture of single labeled and/or dual labeled probes). In some embodiments, each probe can comprise multiple labels (e.g. two labels, three labels, four labels, five labels, six labels, etc). In some embodiments, one or more probes may comprise a single label and one or more probes may comprise multiple labels. In some embodiments, one or more of the probes can be unlabeled and one or more probes may comprise one or more labels.

In some embodiments, the label or labels can be determined directly. In some embodiments, the label or labels can be determined indirectly. In some embodiments, some of the labels can be determined directly and some determined indirectly. Determining a label directly involves determining a property of the label without use of another molecule/compound. For example, determining a fluorescent label may involve viewing a treated sample using a fluorescent microscope, using a slide scanner or using a flow cytometer. Because it is the fluorescence of the label itself that is being observed/measured in the microscope, scanner or cytometer, the determination is said to be direct.

By comparison, indirect determination involves use of an ancillary molecule/compound that recognizes the label of the labeled probes whereby the ancillary molecule/compound (or a label thereon) is determined as a surrogate for determining the label of the labeled probe. For example, the label can be a hapten like digoxigenin. In general, these methods involve the use of an anti-digoxigenin molecule (antibody) conjugated to a secondary label (e.g. an enzyme like horseradish peroxidase, alkaline phosphatase or a fluorophore like fluorescein).

In practice, some probes used in embodiments of the present invention are chosen to determine a select bacteria in a sample. These are referred to as a [or “the”] “bacteria-directed” probe or probes. By “bacteria-directed” refers to a probe or probes that find with specificity to a target within a bacteria, select bacteria. Moreover, said bacteria-directed probe or probes are said to be “capable of determining a [or “the”] select bacteria in a [or “the”] sample” because said bacteria-directed probe or probes selectively bind to a target within the bacteria so that said select bacteria can be determined (for example by fluorescence microscopy or flow cytometry) based on formation of the probe/target complex. Thus, said bacteria-directed probe or probes are used for determining said select bacteria in said sample.

In some embodiments, the select bacteria is a Gram-positive bacteria (e.g. S. aureus) and said bacteria-directed probe or probes are said to be “capable of determining a [or “the”] select bacteria in a [or “the”] sample” or more specifically for S. aureus; “capable of determining S. aureus bacteria in a [or “the”] sample”. In some embodiments, other select bacteria (including as appropriate one or more Gram-negative bacteria) may be selected for determination. In some cases, the sample is also contacted with one or more additional bacteria-directed probes for each select bacteria sought to be determined by practice of the method. Often, the determination of multiple select bacteria in a sample is accomplished by use of a multiplex assay wherein each different type of bacteria is stained with a unique stain, combination of stains and/or unique combination of stain and cell morphology.

The probe or probes chosen to determine a select bacteria (i.e. the bacteria-directed probe or probes) can be a rRNA-directed probe or probes. Said rRNA-directed probe or probes bind with specificity to a target in the rRNA of the select bacteria. The bacteria-directed probe or probes may also be directed to other regulatory RNAs (e.g. messenger RNA (mRNA), small RNA (sRNA) or antisense RNA (aRNA)) or chromosomal DNA or plasmid of a bacteria that are specific to said bacteria. Moreover, the bacteria-directed probe or probes need not be hybridization probes. For example, the bacteria-directed probe or probes can be, for example, antibody-based (See for example: U.S. Pat. No. 6,231,857 and U.S. Pat. No. 7,455,985) since it is known that antibodies can be used to distinguish one type of bacteria from another or others.

As noted several times previously, the methods described herein can be practiced in multiplex mode whereby, for example; 1) two or more select bacteria are determined in a single sample; 2) two or more subsets of select bacteria are determined in a single sample; or 3) two or more select bacteria and two or more subsets of select bacteria are determined in a single sample. In general, such multiplex assays are performed by contacting the sample with additional probes as needed to determine the additional select bacteria and/or subsets of select bacteria. In some embodiments, said contacting can be done simultaneously so that all the different bacteria can be determined at the end of a single procedure. For this embodiment, the probe or probes directed to each different select bacteria can be independently detectable. In general, the labels of the various probes used in practice of the method are selected to produce different stained bacteria based on the type of bacteria. In some cases however, it will be possible to have some identically stained bacteria, whereby one or more of the select bacteria is determined based on morphology of the bacteria (possibly in combination with a determination of the stain).

Rather than multiplex with different (independently detectable) labels (or uniquely stained bacteria), it is also possible to get multiple results by use of a reprobe cycling method (See: US Pat. Application No. 2005/0123959). In a reprobe cycling method, a result is obtained and then the sample is reanalyzed for determining a second, third, fourth, fifth, etc. result. Typically, in a reprobe cycling method, the prior result is removed (erased) before the sample is treated to obtain the next result.

Targets:

In general, a target can be any target molecule (or a portion thereof) that is present within the bacteria during the whole-cell assay that can be determined using a respective probe. Some non-limiting examples of targets include nucleic acid sequences present (e.g. select sequences within rRNA, mRNA, chromosomal DNA or plasmid DNA) within any nucleic acid of the bacteria, an antigen, an antibody, a protein, a peptide and/or a hormone.

In some embodiments, the methods disclosed herein are practiced with one bacteria-directed probe or probes capable of determining a select bacteria that may be present in a sample.

It is to be understood that the methods disclosed herein can be used to determine additional target(s) (for example by multiplexing or reprobe cycling) that might be of interest in a sample and determined during practice of the methods disclosed herein. For example, it is possible to obtain additional information from the sample by contacting said sample with one or more additional probes directed to said additional target(s) whose presence within bacteria of the sample is indicative of an another condition of interest (for example another condition of clinical interest for proper diagnosis of a patient). Said additional condition of interest may be the presence of another bacteria in the sample. Said additional condition of interest may be the presence of yeast in the sample. Said additional condition of interest may be the presence of a plasmid in the select bacteria and/or in other bacteria of the sample. Said additional conditions may be virulence or antibiotic resistance. The method disclosed herein can be used in combination with numerous probes for numerous targets. Accordingly, it is possible by practice of methods disclosed herein to determine one or more additional conditions of interest based on a proper selection of targets (and the respective probe or probes for each target).

Persons of skill in the art will be able to design select suitable targets (and design appropriate probes to said suitable targets) using routine experimentation and commercially available materials and/or information. For example, ISH is commonly used to determine select bacteria (See: Amann, R., “Methodological Aspects of Fluorescence In Situ Hybridization”, Bioscience Microflora, 19(2): 85-91 (2000) and Pemthaler et al., “Fluorescence in situ Hybridization (FISH) with rRNA-targeted Oligonucleotide Probes”, Methods in Microbiology, 30: 207-226 (2001)) including S. aureus bacteria (See: U.S. Pat. No. 6,664,045 at FIG. 3 and US Pat. Application No. 2008/0008994; Cerqueira et al., “DNA Mimics for the Rapid Identification of Microorganisms by Fluorescence in situ Hybridization (FISH)”, Int. J. Mol. Sci., 9: 1944-1960 (2008). As previously noted, targets for such determinations can, for example, be rRNA. This is not intended to be a limitation however, as the target for selecting a bacteria can, for example, be a surface antigen (U.S. Pat. No. 7,455,985).

Forming Probe/Target Complexes:

The select bacteria are determined by determining formation of the appropriate probe/target complexes within the bacteria of the sample. In brief, by contacting the sample with probes chosen for their affinity for their respective targets known to be associated with (and specific for) the select bacteria, the appropriate probe/target complexes will form within the bacteria of the sample.

The nature of the probe/target complex is determined by the nature of the probe and its respective target. Various types of probe/target complexes are contemplated. For example, hybridization probes for bacteria determination can be rRNA-directed or mRNA-directed. Thus, each complex formed upon binding of the probe to its target is a probe/rRNA complex or probe/mRNA complex, respectively. Similarly, hybridization probes can be chromosome DNA-directed, or plasmid-directed. Hence, each complex formed upon binding of the probe to its respective target is a probe/chromosome DNA complex or probe/plasmid complex, respectively. With respect to antibody probes, binding of the antibody to its antigen target produces an antibody/antigen complex.

Those of skill in the art will recognize that the probe/target complexes in the bacteria are formed under suitable binding conditions (or more correctly termed “suitable hybridization conditions” for hybridization probes). Suitable binding conditions for each probe/target complex will be determined based on the nature of the probe and target. It suffices to say that suitable binding conditions are reflected in conditions where the interactions of the probe and its respective target are specific. Moreover, persons of ordinary skill in the art can determine suitable binding conditions for forming many types of probe/target complexes. Indeed, numerous hybridization buffers are commercially available for use in various assay formats. It is to be understood that binding conditions need not be completely optimized but rather that the conditions merely be suitable for specific binding of the probe to its respective target such that the assay produces accurate and reproducible result. Moreover, where different types of probes (e.g. hybridization probes and antibody-based probes) are used in the same contacting step, binding conditions should be suitable for the binding of each type of probe to its respective target.

Determining Probe/Target Complexes:

Once formed, the probe/target complexes can be determined. The probe/target complexes can be determined using a label associated with each different (or different type of) probe/target complex. In some embodiments, all labels associated with different (or different types of) probe/target complexes are the same. In some embodiments, different labels (or combinations of labels) are associated with each different (or different type of) probe/target complex. In some embodiments, there is a mixture of the same label associated with some of the different (or different types of) probe/target complexes and different labels associated with others of the different (or different types of) of probe/target complexes.

A probe/target complex can be determined directly or indirectly. By “directly” means that the probe of the probe/target complex comprises a linked label which label is determined based on its own properties. By “indirectly” means that the probe/target complex is determined using a secondary composition (e.g. a labeled antibody) that comprises a label and that binds to (or interacts with) the probe/target complex (or a label linked to the probe/target complexes), wherein said label is determined as indicia of the probe/target complex. Regardless, determining the label correlates with determining the probe/target complex.

In whole-cell assays, determining the probe/target complexes can, in some embodiments, be performed by examining how the cells (i.e. the bacteria) are stained. In brief, regardless of whether the labeling is direct or indirect, the cells become stained because the label(s) associated (directly or indirectly) with the probe/target complex or complexes is/are assimilated within (or at least on the surface of) the intact cells (i.e. bacteria). As noted previously, it is possible to use unique labels and/or unique combinations of labels for different bacteria and/or traits. Thus, any method capable of determining the stained bacteria in the sample can be used to determine the select bacteria.

For example, the select bacteria can be determined based on their visual appearance under a microscope. In some embodiments, the process can be automated so that the result can be determined using a computer and algorithm. In some embodiments, the select bacteria can be determined using a slide-scanner. Similarly, a slide scanner can be automated so that the result can be determined using a computer and algorithm. In some embodiments, the select bacteria can be determined using a flow-cytometer. Likewise, a flow-cytometer can be automated so that the result can be determined using a computer and algorithm. Moreover, any other instrument or method suitable for determining stained cells can be used to determine the probe/target complexes formed using the inventive methods disclosed herein.

Hybridization Conditions/Stringency:

Persons of ordinary skill in the art will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Blocking probes may also be used as a means to improve discrimination beyond the limits possible by mere optimization of stringency factors. Optimal stringency for forming a probe/target complex is often found by the well-known technique of fixing several of the aforementioned stringency factors and then determining the effect of varying a single stringency factor. The same stringency factors can be modulated to thereby control the stringency of hybridization of a nucleic acid mimic, nucleic acid analog or chimera to a nucleic acid target (e.g. a sequence within rRNA, mRNA or chromosomal DNA), except that for some of these modified oligomers (e.g. PNA) the hybridization may be fairly independent of ionic strength. Optimal or suitable stringency for an assay may be experimentally determined by examination of each stringency factor until the desired degree of discrimination is achieved. Nevertheless, optimal stringency is not required. Rather, all that is required is that the non-specific binding of probes to other than their respective targets is minimized in the assay to the extent necessary to achieve an accurate and reproducible result. Other conditions include the use of aqueous alcohol solutions (which may also be used for combined fixation) (US20070128646).

The use of buffered saline, such as but not limited to 0.75 M NaCl, 5 mM EDTA, 0.10 M Tris HCl, pH 7.8 (previously used for algae (PLoS ONE 6(10): e25527.

doi:10.1371/journal.pone.0025527), as a non-toxic hybridization buffer for bacteria and in particularly Gram-positive bacteria, such as S. aureus, as exemplified in Example 2, is also within the embodiment of this invention.

As time to result can be an important factor particularly for clinical samples, the hybridization reactions performed in the examples provided below differ significantly from those of Matsuhisa et al., (Biotech Histochem. 69:31-7.), inter alia, in that they were performed in less than 2 hours rather than over-night.

Fixing:

Whole cell assays can be performed using fixed cells. Fixing is the process of treating samples to thereby preserve and/or prepare said cells for analysis. Fixed samples can be stored for a period time before they are analyzed. A commonly used fixative reagent is paraformaldehyde. Other commonly used fixative reagents include glyoxal, glutaraldehyde, zinc salts, heat, alcohols (methanol and ethanol), acidic solutions and combinations of any two or more of these. In some embodiments, methods disclosed herein can be practiced by contacting the sample with a fixative reagent or reagents. A commonly used process for fixing cells is referred to as flame fixation or heat fixation; which process may (or may also not) be accompanied by contacting the cells with a reagent or reagents. Thus, the methods disclosed herein can be practiced with a fixation step which may (or may not) include contacting the sample with a reagent or reagents. Any fixative reagent or reagents may contain other compositions not strictly related to fixation. For example, in some embodiments one or more probes may be added to a fixation reagent or reagents. In this way, fixation and probe/target formation can be performed simultaneously. Any combination of reagents is permissible so long as the combination operates for its intended purpose much in the way that the individual reagent or reagents would if not combined.

Permeabilizing or Lysis:

Permeabilization or lysis of cells is the process by which the cell membrane/cell wall is modified so that reagents required to perform an assay can gain access to the target. Some non-limiting examples of cell permeabilizing or lysis reagents include solutions/formulations comprising one or more enzymes such as lysozyme, and proteinases (e.g. proteinase-K and/or achromopeptidase). To permeabilize the cells, said enzymes can be contacted with the sample and thereby partially digest the cell membrane and/or cell wall. In some embodiments, the cell permeabilizing or lysis reagents are chemicals, mixtures of chemicals and enzymes or sequential treatment with chemical(s) and enzyme(s) in any order.

The degree of permeabilization or lysis depends on the nature of the reagents that must penetrate into the cell for practice of the particular assay. Generally, as the size of the molecule that must pass through the cell membrane/cell wall increases, a greater the degree of permeabilization must be performed. Permeabilization or lysis may also be performed by other mechanisms, such as heating, (ultra)sound, or mechanical forces and is within the embodiment of the invention.

### Various Embodiments of the Invention

It should be understood that the order of steps or order for performing certain actions is immaterial so long as the present teachings remain operable or unless otherwise specified. Moreover, in some embodiments, two or more steps or actions can be conducted simultaneously so long as the present teachings remain operable or unless otherwise specified.

This invention pertains, inter alia, to methods for determining select bacteria of a sample, where said bacteria are located intracellular. It is to be understood that the methods described herein are not limited to determining one select bacteria. Rather, the methods can be used to determine multiple bacteria in a sample, incl. subsets of the same bacteria of the sample. In some embodiments, the multiple bacteria and/or multiple subsets of select bacteria will be determined using a multiplex assay. The multiplex assay can involve the use of differential staining of the bacteria whereby the different stain or stains a bacteria exhibits is used to determine the different bacteria and/or subset(s). Therefore, in some embodiments, this invention pertains to a method comprising contacting a sample with a bacteria-directed probe or probes capable of determining a select bacteria in the sample. Often the sample will be suspected of comprising one or more bacteria. In some embodiments, the sample is treated with a permeabilizing or lysis reagent, whereas in other embodiments a permeabilization or lysis reagents is not used. It is understood that the use of permeabilization or lysis reagents may be directed towards permeabilization or lysis of the bacteria and/or the cells containing the bacteria. In some embodiments, the hybridization buffer contains a denaturing reagents, such as formamide, whereas in other embodiments a hybridization buffer does not contain a denaturing reagent. Hybridization buffer without denaturing reagents, such a formamide, may be buffered saline, such as but not limited to NaCl, Tris and EDTA, i.e. 0.75M NaCl, 5 mM EDTA, 0.1M Tris, pH 7.8. In some embodiments, signal amplification is used to increase the signal prior to determining the bacteria, whereas in other embodiments signal amplification is not used. In preferred embodiments, the method is performed without permeabilization or lysis reagents, and/or without hybridization buffer containing denaturing chemicals and/or without the use of signal amplification. In yet another preferred embodiment, the method is performed without permeabilization or lysis reagents and without signal amplification.

According to these various methods, determination of the select bacteria involves determining the formation of probe/target complexes for the bacteria-directed probe or probes and chromosomal DNA-, RNA- and/or plasmid-directed labeled probe or probes, respectively. The formation of the probe/target complexes is accomplished under suitable binding conditions (or suitable hybridization conditions as appropriate). In some embodiments, formation of the respective probe/target complex or complexes will be evident based on the nature of the staining of the bacteria. Thus, for these embodiments, the select bacteria and/or subsets can be determined by analysis of the staining of individual bacteria. The staining of individual bacteria can, for example, be monitored (determined) using a microscope, slide scanner or flow cytometer.

In some embodiments, bacteria-directed probe or probes is/are antibody-based. As such, the target for each probe is an antigen found on the surface of, or within, the select Gram-positive bacteria.

In some embodiments, more than one select bacteria can be determined. In some embodiments, this can be accomplished by multiplexing. In some embodiments, this can be accomplished by reprobe cycling the sample. In some embodiments, this can be accomplished by both multiplex and reprobe cycling the sample. Thus, in some embodiments, these methods further comprises contacting the sample with a second bacteria-directed probe or probes capable of determining a second select bacteria in the sample. It is to be understood that the method can also be practiced by contacting the sample with additional probes or probe sets to one or more additional select bacteria and/or subset of select bacteria.

The method is practiced on substantial intact bacteria present intracellular by contacting the sample with one or more bacteria-directed probes comprising labels capable of determining the bacteria in said sample; and determining the one or more bacteria.

In some embodiments, these methods can be practiced using only mRNA-directed probe or probes wherein said probe or probes are capable of determining mRNA associated with methicillin-resistance. In some embodiments, only a single mRNA-directed probe is used to determine methicillin-resistance. In some embodiments, two or more mRNA-directed probes are used to determine methicillin-resistance (i.e. a mixture of mRNA-directed probes which probes can each be labeled with one or two labels).

In some embodiments, bacteria-directed probe or probes is/are antibody-based. As such, the target for each probe is an antigen found on the surface of, or within, the select bacteria.

In some embodiments, the bacteria-directed probe or probes is/are rRNA-directed. As such, the target for each probe is a nucleobase sequence found within rRNA of the select bacteria. In some embodiments, the bacteria-directed probe or probes is/are directed to other regulatory RNA (e.g.

sRNA or aRNA). As such, the target for each probe is a nucleobase sequence of (or within) mRNA or regulatory RNA (e.g. sRNA or aRNA), respectively.

In some embodiments, all labels are fluorescent labels and said method is a fluorescent in-situ hybridization (FISH) assay. In some embodiments, a label or labels of said DNA and/or RNA-directed labeled probe or probes is/are determined directly. In some embodiments, the DNA-and/or RNA-directed labeled probe or probes is/are PNA. In some embodiments, the DNA-and/or RNA-directed labeled probe or probes is/are 10 to 20 nucleobase subunits in length. In some embodiments, signal amplification is used to directly or indirectly amplify signal of a label or labels of said DNA and/or RNA-directed labeled probe or probes.

In some embodiments, the method can be practiced without treating the sample with a cell permeabilizing or lysis reagent or reagents.

In some embodiments, the method can be practiced where the samples is immobilized onto a solid support, such as a microscope slides, filter or bead, whereas in other embodiments, the method is practiced where the sample remain in solution.

In some embodiments, the host cells are predatory host cells, such as phagotytic cells, degrading the intracellular bacteria, whereas in other embodiments the host cells and the intracellular bacteria are living in symbiosis. The intracellular bacteria may therefore be viable (alive) or non-viable (dead).

In summary, there are many different ways to practice the method of the invention of which preferred embodiments can be described by:

A method for the determination of one or more intracellular select bacteria, said method comprising:
a. providing a sample comprising host cells enclosing the intracellular bacteria;
b. fixing the sample to maintain the intracellular bacteria substantially intact;
c. contacting the sample with one or more select bacteria-directed probes comprising labels capable of determining the one or more select bacteria in said sample; and
d. determining the one or more bacteria;

and said method not comprising:
e. contacting with a cell permeabilizing or cell lysing reagent; and/or
f. signal amplification of said label; and/or
g. contacting with a denaturing reagent.

The method above may be practiced as an in situ hybridization method where the host cells are phagocytic cells (predatory host cells), and/or the bacteria is S. aureus, and/or the bacteria-directed probes are PNA probes, and/or the labels are fluorescent, and/or the duration of the contacting is less than 2 hours (i.e. 1, 5, 15, 30, 60 or 90 minutes), and/or the hybridization buffer is non-toxic, and the sample is either attached to a solid support or in solution.

### EXAMPLES

Aspects of the present teachings can be further understood in light of the following examples, which should not be construed as limiting the scope of the present teachings in any way.

### Example 1

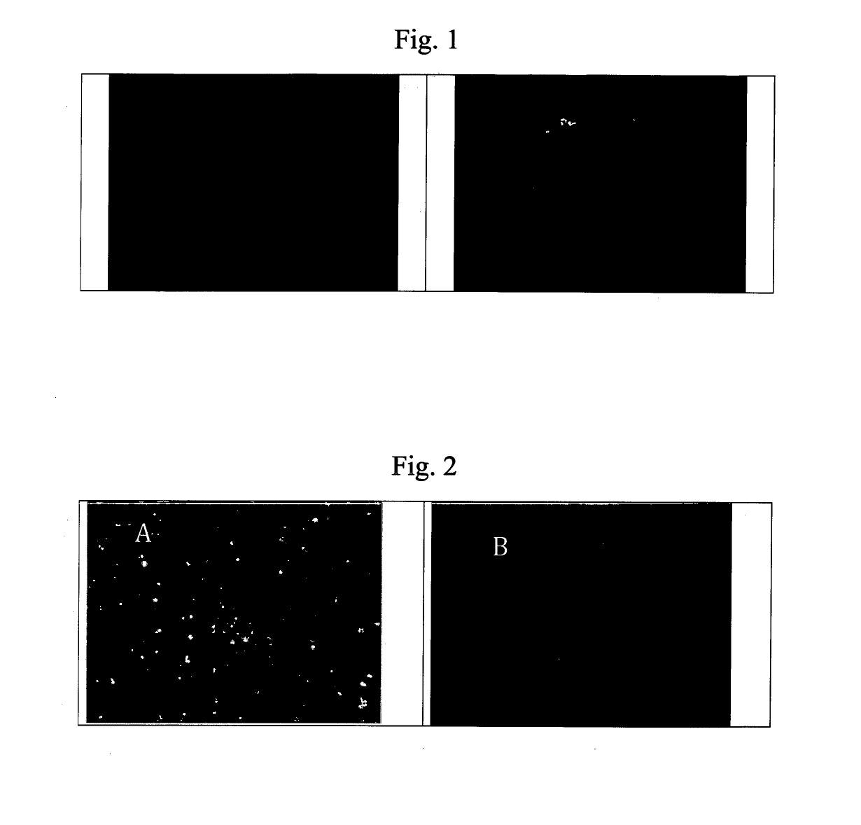

Staphylococcus aureus were experimentally spiked into donor blood and incubated 15-45 min for ingestion of S. aureus by phagocytic cells. The PMN fraction was purified and subsequent analyzed by S. aureus PNA FISH on microscope slides in accordance with Oliveira et al., J. Clin. Microbiol 40:247-251 (2002). Briefly, the samples was fixed onto microscope slides and hybridized with fluorescein-labeled PNA probes in hybridization buffer containing formamide for 30 minutes. Unbound PNA probes were removed by stringent wash for 30 minutes at 55° C. and counterstained with DAPI for visualization of both PMNs (blue) and S. aureus (blue). Examination by flourescence microscopy showed PMNs, including S. aureus (A) using DAPI (blue) filter and only S. aureus (B) using FITC/Texas Red filter. (blue cocci are observed within the PMN with the same morphology and position as the green cocci), See FIG. 1. In conclusion, intracellular bacteria in phagocytic cells can be determined by fluorescence in situ hybridization using PNA probes without the use of permeabilization or lysis reagents and without using signal amplification.

### Example 2

S. aureus and Staphylococcus epidermidis were analyzed by fluorescence in situ hybridization in accordance with PLoS ONE 6(10): e25527 modified with PNA probe sequence and temperature (55° C.) from J. Clin. Microbiol 40:247-251 (2002). Briefly, the bacteria were fixed in solution using saline ethanol and washed twice (pre-hybridization) with hybridization buffer (0.75 M NaCl, 5 mM EDTA, 0.10 M Tris HCl, pH 7.8) followed by hybridization with PNA probe for 1 hour at 55° C. Unbound probe was removed by washing and the samples were mounted onto microscope slides. Examination by flourescence microscopy using FITC/Texas Red filter showed strong fluorescence of S. aureus (A) and low/none fluorescence of S. epidermidis (B), See FIG. 2. In conclusion, S. aureus can be determined by fluorescence in situ hybridization using PNA probes in buffered saline as hybridization buffer and without immobilizing the bacteria on microscope slides during the hybridization.

1. A method for whole cell analysis of bacteria for the determination of one or more intracellular select bacteria, said method comprising:
a. providing a blood sample comprising phagocytic cells enclosing the intracellular bacteria;
b. fixing the sample to maintain the intracellular bacteria substantially intact;
c. contacting the sample with one or more select bacteria-directed probes comprising labels capable of determining the one or more select bacteria in said sample; and
d. determining the one or more bacteria in said phagocytic cells.

2. The method of claim 1, wherein the labels are fluorescent labels and/or the bacteria-directed probes are PNA probes.

3. The method of claim 1, wherein the contacting is performed in less than 2 hours.

4. (canceled)

5. The method of claim 1, where the method is practiced without the use cell permeabilizing or lysing reagent(s) and/or step(s).

6. The method of claim 1, where said labels are not signal amplified.

7. The method of claim 1, where said blood sample is fractionated blood.

8. (canceled)

9. (canceled)

10. The method of claim 1, wherein said bacteria are S. aureus

11. The method of claim 1, wherein the select bacteria-directed probes are directed towards rRNA, mRNA, sRNA or aRNA.

12. The method of claim 1, wherein the select bacteria-directed probes are directed towards rRNA, mRNA, sRNA or aRNA, wherein the labels are fluorescent labels and/or the bacteria-directed probes are PNA probes, and wherein the contacting is performed in less than 2 hours.
